(19) United States
(12) Patent Application Publication
(10) Pub. No.: US 2022/0093004 A1
(43) Pub. Date: Mar. 24, 2022

(54) DENTAL HYGIENE TOY AND TRAINING KIT

(71) Applicant: CHANTEL WALKER, HOUSTON, TX (US)

(72) Inventor: CHANTEL WALKER, HOUSTON, TX (US)

(21) Appl. No.: 17/402,580

(22) Filed: Aug. 15, 2021

Related U.S. Application Data
(60) Provisional application No. 63/082,454, filed on Sep. 23, 2020.

(51) Int. Cl. G09B 19/00; A46B 9/04; A61C 15/04; A61B 17/24; A63H 3/00; A63H 3/48; A63H 3/44; A63H 3/52

(52) U.S. Cl. CPC G09B 19/0084; A46B 9/04; A61C 15/046; A61B 17/244; A61C 2203/00; A63H 3/48; A63H 3/44; A63H 3/52; A63H 3/003

(57) ABSTRACT

The inventive concept is a dental hygiene toy kit assembly and method that includes a polymer doll member with a jaw member pivotally coupled at a distal end of the jaw member to a lower portion of the doll's head designed to be opened and closed. When open, the jaw member exposes a lower row of protruding teeth members, an upper row of protruding teeth members, and a tongue member disposed on an upper portion of the jaw member interior to the head assembly. A toothbrush, tongue scraper, and flosser, each with a widened handle, is included to perform cleaning operations on the teeth and tongue members. A substantially cuboid, divided housing assembly is designed to be in an open or close state, a foldable, toy dental chair in an unfolded state configured to seat the doll member when the housing assembly is in an open state.

### CLAIM OF PRIORITY

This application claims priority to and the benefit of U.S. Provisional application with Ser. No. 63/082,454, filed on Sep. 23, 2020, with the title Dental Hygiene Training Toy, which is incorporated herein by reference in its entirety.

### FIELD OF THE INVENTION

The inventive concept relates generally to a dental hygiene training toy and training kit for a user.

### BACKGROUND

Currently, there are a number of solutions for educational toys on healthcare. One of these solutions attempts to use standard dolls, but this solution fails to meet the needs of the market because dolls are not specialized for health training. Another solution attempts to use books and videos, but this solution is unable to meet the needs of the market because kids cannot play with books and videos interactively to develop their physical hygiene cleansing skills. Still another solution seeks to use play sets with healthcare tools, but this solution also fails to meet market needs because the tools may become non-hygienic with repetitive use. Therefore, there currently exists a need in the market for an improved apparatus that demonstrates proper dental cleaning and hygiene.

### SUMMARY OF THE INVENTION

The inventive concept is a dental hygiene training toy kit assembly and training method that includes a polymer doll member, the doll member having a body, two arms, and two legs, and a head assembly. In a related embodiment, the doll member need not include any appendages. A jaw member of the inventive concept has a distal end and a proximal end. The distal end of the jaw member is pivotally coupled to the head assembly at a lower front face of the head assembly to create a movable jaw assembly. The movable jaw assembly is adapted to be in an open or closed state, the proximal end of the movable jaw assembly contiguously coupled to a proximal edge of the lower front face of the head assembly. The jaw member, when in an open state, exposes a lower row of protruding teeth members disposed on an inner edge of the proximal end of the jaw member, and exposes an upper row of protruding teeth members disposed on an inner edge of the proximal edge of the lower front face of the head assembly, and exposes a tongue member disposed on an upper middle and distal portion of the jaw member and interior to the head assembly. At least one or more of a toothbrush, tongue scraper, and floss tool, each with a widened handle, is designed to be used by a user exemplified by a child to perform cleaning operations on the teeth and tongue members. A substantially cuboid, divided housing assembly is designed to be in an open or close state. A foldable, toy dental chair is disposed within the housing assembly when the housing assembly is in a closed state. The toy dental chair is in an unfolded state when the housing assembly is in an open state and is so configured to seat the doll member.

In one embodiment of the dental hygiene training toy kit assembly, a ridged silicone grip member is disposed on an outer surface of the widened handle members of the toothbrush, tongue scraper, and floss tool.

In one embodiment of the dental hygiene training toy kit assembly, a substantially planar mat member affords a playing surface circumscribed by a raised ridge member, the mat member providing a playing surface for the dental hygiene training kit assembly, the playing surface further displaying image members directing where to place other members of the kit assembly, the playing surface further adapted for the child to play on the mat member with other members of the kit assembly.

In one embodiment of the dental hygiene training toy kit assembly, the mat member is machine washable.

One of ordinary skill in the art would recognize that the widened handle could be narrowed or otherwise designed with grip assemblies that make the handle easier to use.

In one embodiment of the dental hygiene toy kit assembly, a handle portion is pivotally coupled to the housing assembly.

In one embodiment of the dental hygiene toy kit assembly, a mouthwash bottle is included in the kit assembly.

In one embodiment of the dental hygiene toy kit assembly, a removable hair member is coupled to an upper portion of the head assembly, the kit designed to include one or more hair members. In one embodiment of the dental hygiene training toy kit assembly, the removable hair member is coupled to an upper portion of the head assembly via at least one snap member, the snap member having a substantially concave receiving portion disposed on the head assembly or on the hair member and a protruding connector member disposed on the opposing head assembly or hair member adapted to be frictionally coupled within the concave receiving portion, the concave receiving portion and the protruding connecting member further being made substantially from silicone.

The inventive concept may include other features common among dolls such as changes in clothes, changes in hair styles, ethnicity, and accessories such as shoes, purses, or backpacks.

It would be advantageous to have a dental hygiene toy kit assembly that is interchangeable. Furthermore, it would be advantageous to have a dental hygiene toy kit assembly that is portable. Still further, it would be advantageous to have a dental hygiene toy kit assembly that is foldable.

The inventive concept advantageously fills the aforementioned deficiencies by providing a dental hygiene training toy, which provides a fun way to learn about dental hygiene. The dental hygiene toy kit assembly has a demonstration doll and a play mat. The dental hygiene toy kit assembly fulfills the need for a dental educational toy.

Among other things, it is an advantage of the inventive concept to provide a dental hygiene toy that does not suffer from problems or deficiencies associated with prior solutions. It is still further an advantage of the inventive concept to be interchangeable. Still further, the dental hygiene toy kit assembly has a folding dentist chair.

The inventive concept now will be described more fully hereinafter with reference to the accompanying drawings, which are intended to be read in conjunction with both this summary, the detailed description, and any preferred and/or particular embodiments specifically discussed or otherwise disclosed. This inventive concept may, however, be embodied in many different forms and should not be construed as limited to the embodiments set forth herein; rather, these embodiments are provided by way of illustration only and so that this disclosure will be thorough, complete and will fully convey the full scope of the inventive concept to those skilled in the art.

### BRIEF DESCRIPTION OF THE DRAWINGS

FIG. 1 illustrates a front view of the dental hygiene toy kit assembly.

FIG. 2 illustrates a back view of the dental hygiene toy kit assembly.

FIG. 3 illustrates a right side view of the dental hygiene toy kit assembly.

FIG. 4 illustrates a left side view of the dental hygiene toy kit assembly.

FIG. 5 illustrates a top view of the dental hygiene toy kit assembly.

FIG. 6 illustrates a bottom view of the dental hygiene toy kit assembly.

FIG. 7 illustrates a perspective view of the dental hygiene toy kit assembly.

FIG. 8 illustrates a view of a head assembly with an open jaw member of the dental hygiene toy kit assembly.

FIGS. 9A and 9B illustrate views of a portable dental case and a set of dental tools of the dental hygiene toy kit assembly, respectively.

FIG. 10 illustrates a view of the changeable hair portion of the dental hygiene toy kit assembly.

FIG. 11 illustrates a view of the dental chair of the dental hygiene toy kit assembly.

FIGS. 12A and 12B illustrate full and cutaway top views, respectively, of a planar mat member which forms part of the kit assembly.

FIG. 13 illustrates a view of the deployed dental hygiene toy kit assembly.

FIG. 14A-14B illustrate a flowchart of a method of teaching dental hygiene via the dental hygiene toy kit assembly.

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

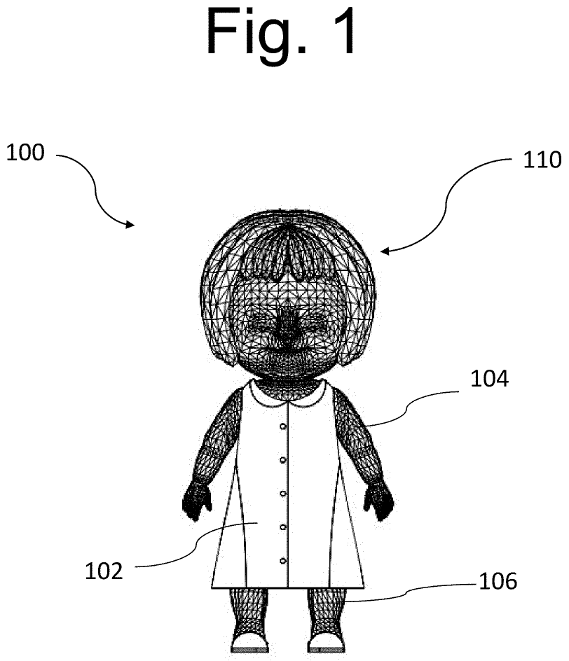

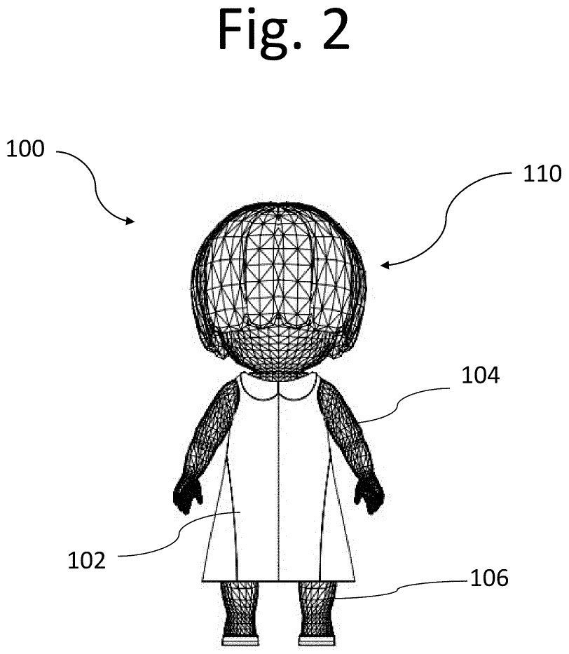

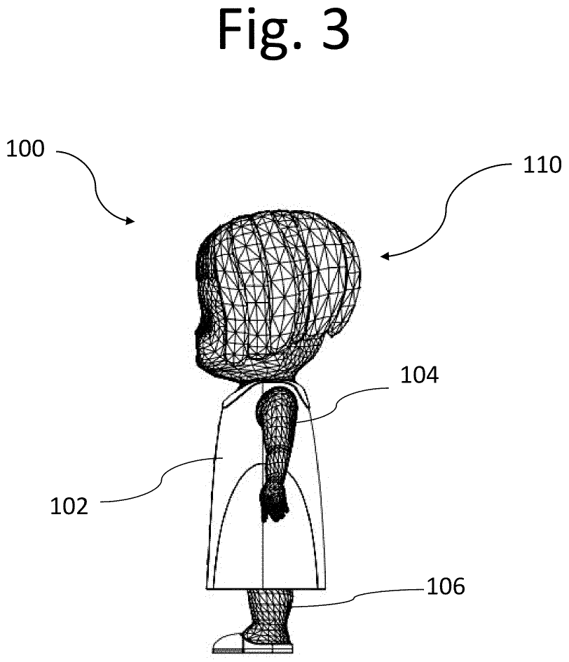

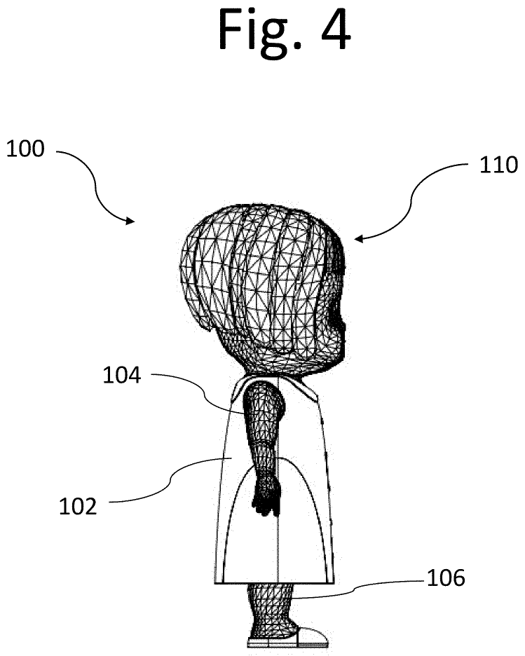

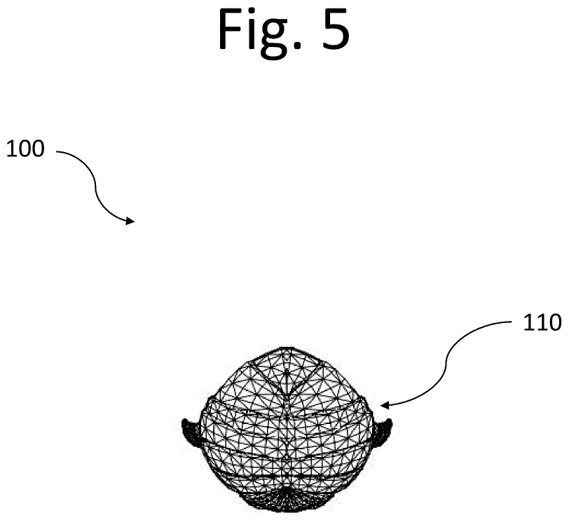

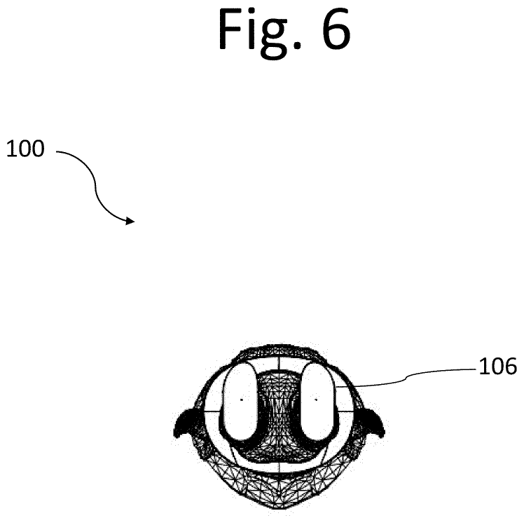

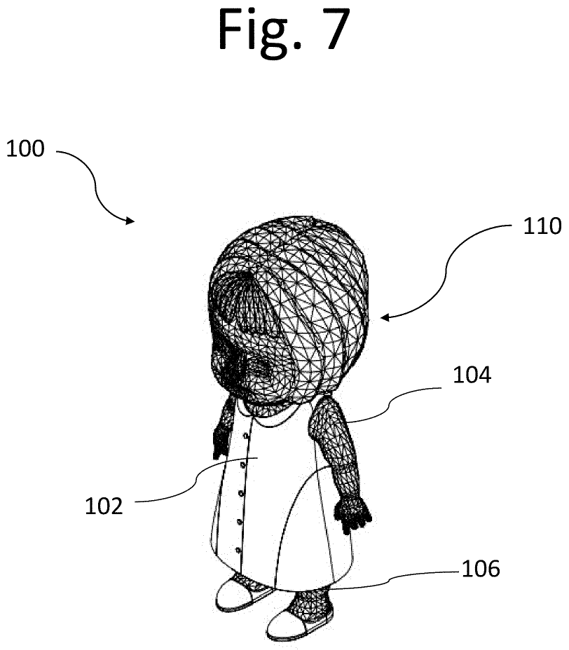

Referring to the figures, FIGS. 1-12B illustrate the inventive concept being a dental hygiene training toy kit assembly 10 that has a polymer doll member 100, the doll member having a body 102, two arms 104, two legs 106, and a head assembly 110. A jaw member 120 of the inventive concept has a distal end of the jaw member 129 and a proximal end of the jaw member 121. The distal end of the jaw member 129 is pivotally coupled to the head assembly 110 at a lower front face of the head assembly 130 to create a movable jaw assembly 140. The movable jaw assembly 140 is adapted to be in an open or closed state, the proximal end of the movable jaw assembly 141 contiguously coupled to a proximal edge of the lower front face of the head assembly 111. The jaw member 120, when in an open state, exposes a lower row of protruding teeth members 122 disposed on an inner edge of the proximal end of the jaw member 124, and exposes an upper row of protruding teeth members 112 disposed on an inner edge of the proximal edge of the lower front face of the head assembly 114, and a tongue member 116 disposed on an upper middle portion of the jaw member 125 and the distal portion of the jaw member 129 and interior to the head assembly 200.

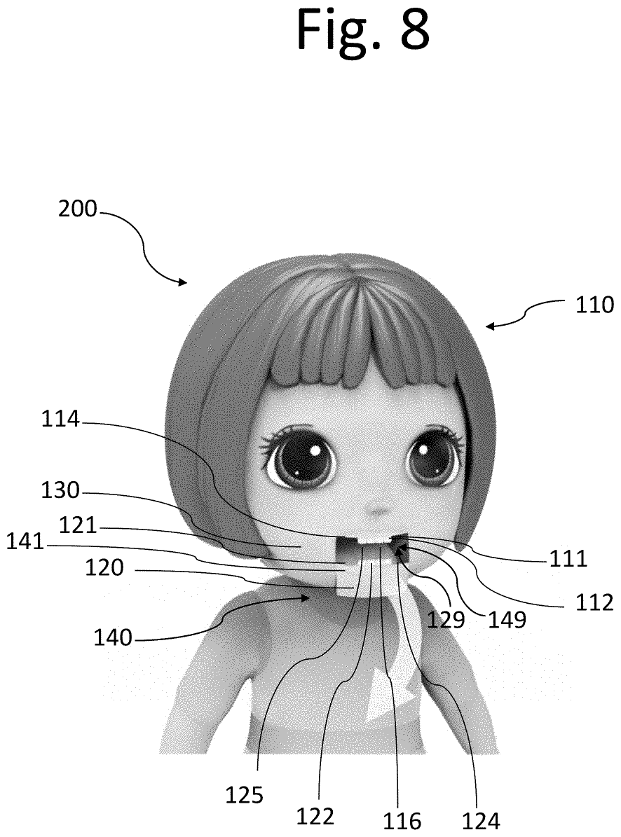

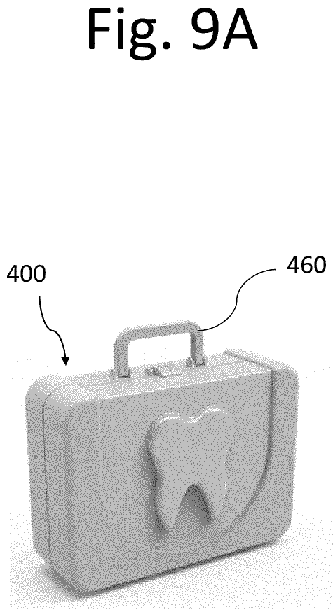

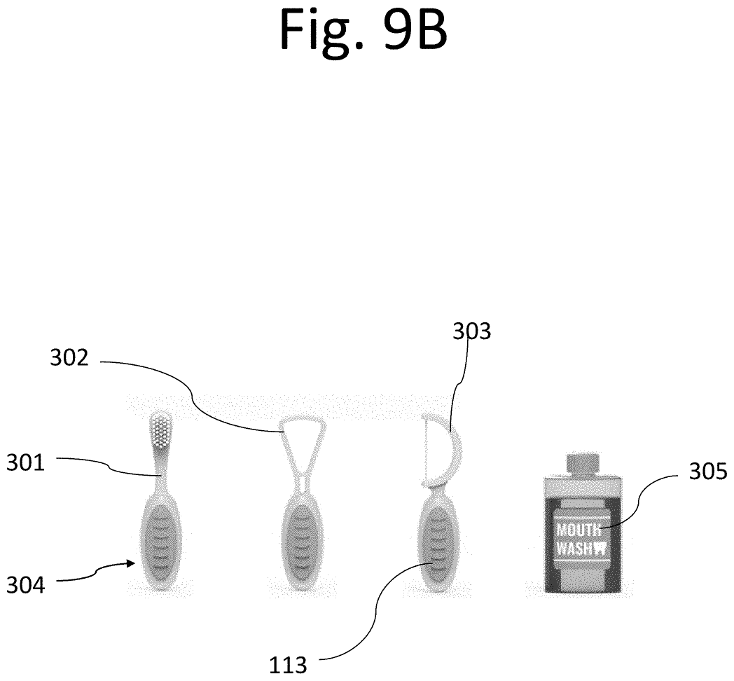

FIGS. 8-9B illustrates an open jaw member of doll head assembly 110 along with dental tools 300 including a toothbrush 301, tongue scraper 302, and floss tool 303, each with a widened handle 304, designed to be used by the child to perform cleaning operations on the teeth and tongue members 112, 116, 122. The dental hygiene training toy kit assembly 10 may include a ridged silicone grip member 113 disposed on an outer surface of the widened handle members 304 of the toothbrush, tongue scraper, and floss tool 112, 116, 122. In one embodiment of the dental hygiene toy kit and training assembly 10, a mouthwash bottle 305 is included in the kit assembly 10. In one embodiment of the dental hygiene toy kit assembly 10, a removable hair member 170 is coupled to an upper portion of the head assembly, the kit designed to include one or more hair members 170.

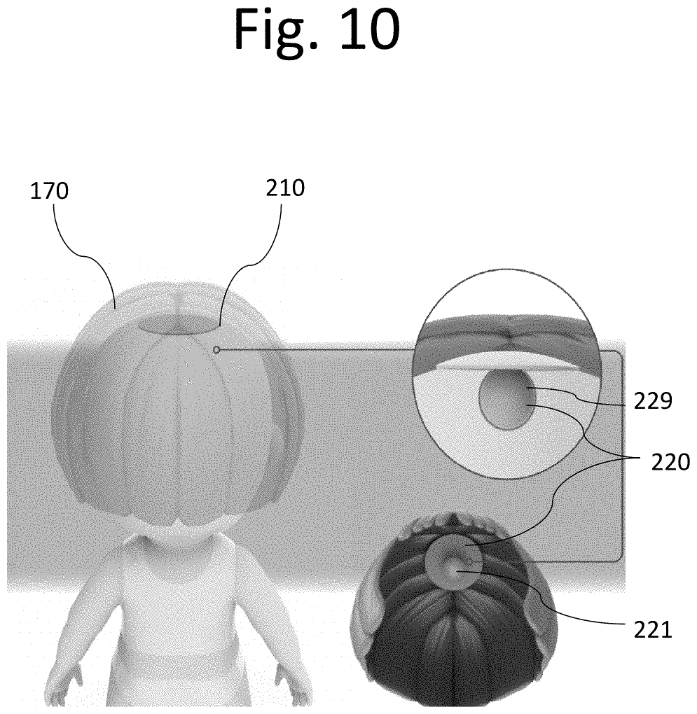

FIG. 10 illustrates that in this exemplary embodiment of the dental hygiene training toy kit assembly 10, the removable hair member 170 is coupled to an upper portion of the head assembly 210 via at least one snap member 220, the snap member 220 having a substantially concave receiving portion 229 disposed on the head assembly 200 or on the hair member 170 and a protruding connector member 221 disposed on the opposing head assembly 200 or hair member 170 adapted to be frictionally coupled within the concave receiving portion 229, the concave receiving portion 229 and the protruding connecting member 221 further being made substantially from silicone.

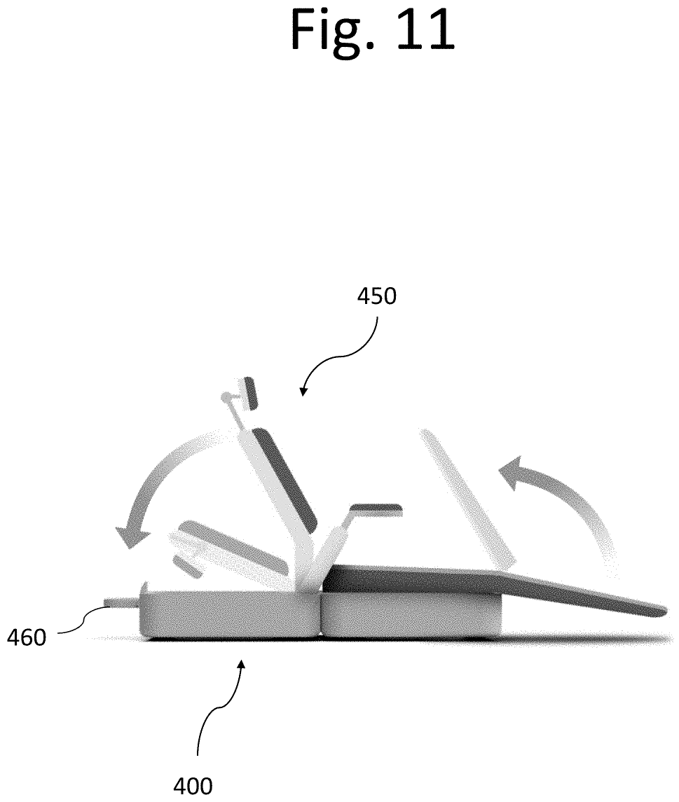

FIG. 11 illustrates that in this exemplary embodiment, a substantially cuboid, divided housing assembly 400 is designed to be in an open or close state, with a foldable, toy dental chair 450 disposed within the housing assembly 400 when the housing assembly 400 is in a closed state, the toy dental chair 450 being in an unfolded state configured to seat the doll member 100 when the housing assembly 400 is in an open state. In this exemplary embodiment of the dental hygiene training toy kit assembly, a handle portion 460 is pivotally coupled to the housing assembly 400.

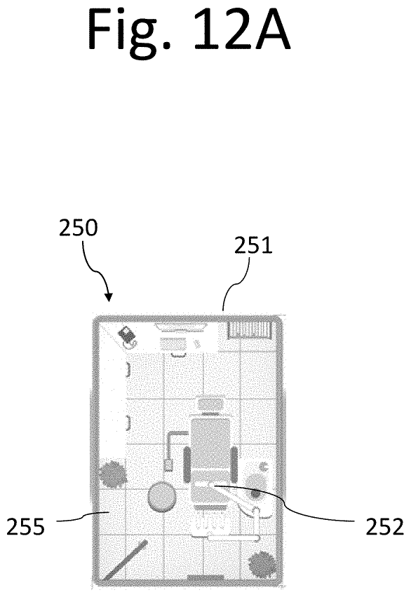

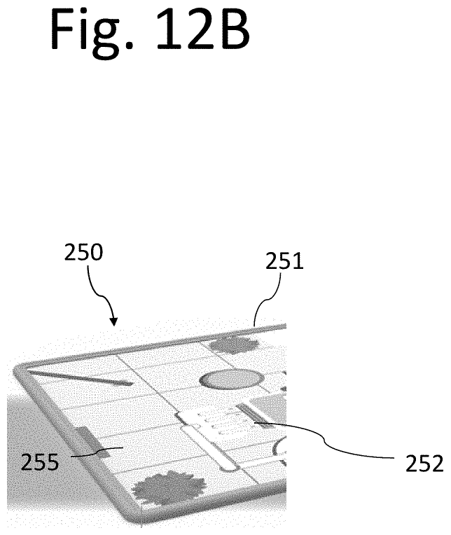

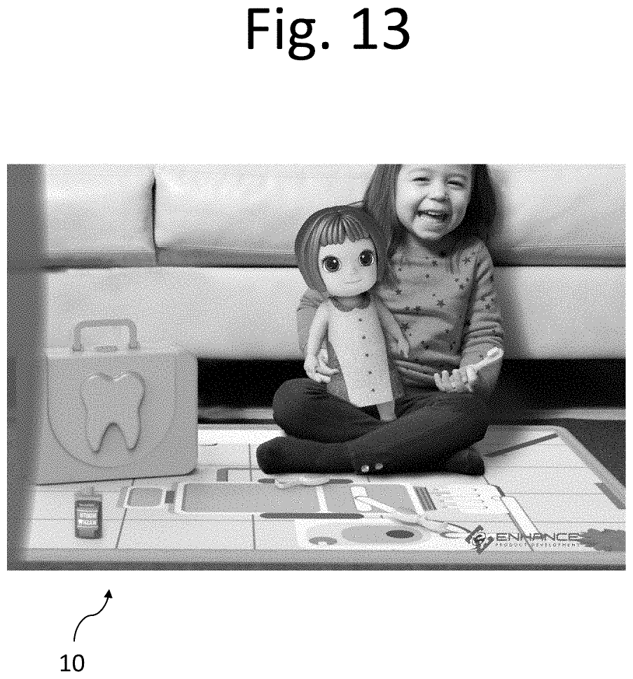

Referring now to FIGS. 12A-12B and 13, there is illustrated full and cutaway top views, respectively, of a planar mat member 250 which forms part of the kit assembly of this embodiment of the dental hygiene training toy kit assembly 10. Substantially planar mat member 250 affords a playing surface 255 for a child (see FIG. 13), circumscribed by a raised ridge member 251, the mat member 250 providing a playing surface 255 for the dental hygiene training kit assembly 10. Playing surface 255 further displays image members 252 directing where to place other members of the kit assembly 10, the playing surface 252 further designed for the child to play on the mat member 250 with other members of the kit assembly 10. In this exemplary embodiment of the dental hygiene training toy kit assembly 10, the mat member 250 is machine washable.

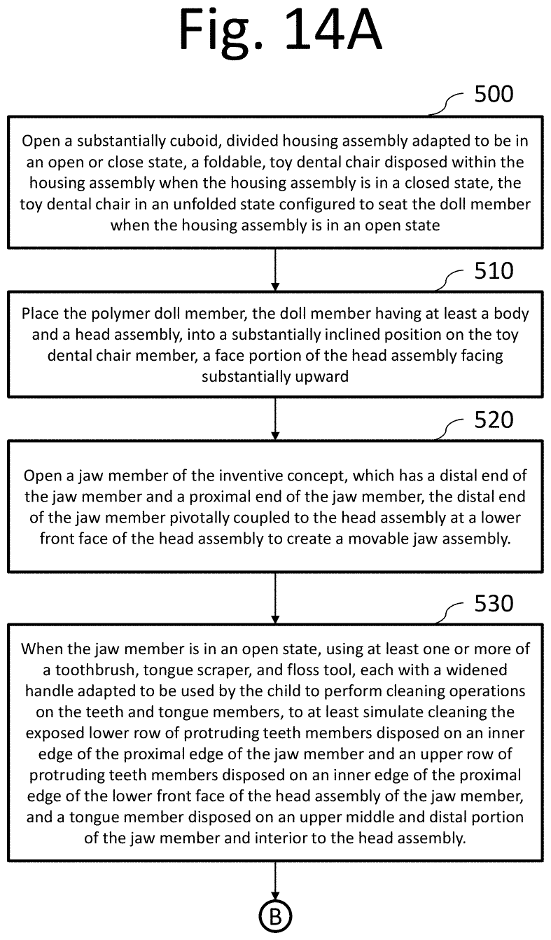

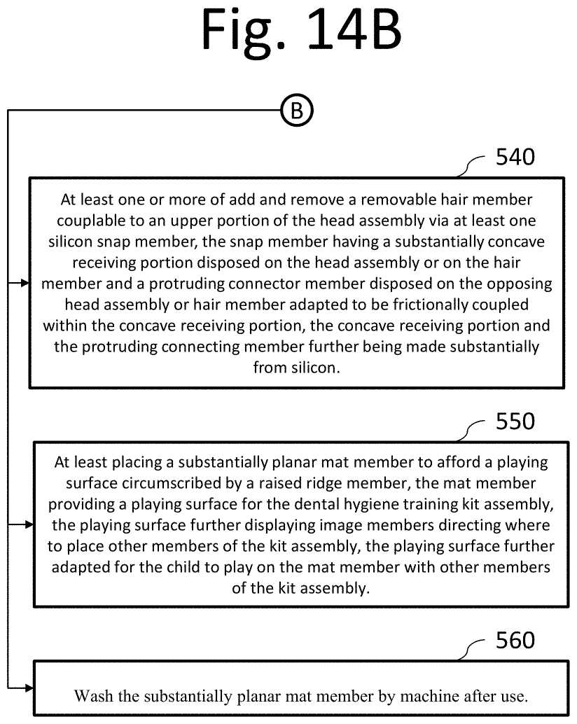

FIGS. 14A-14B illustrate a method 50 of teaching dental hygiene via the dental hygiene toy training kit assembly 10, method 50 including step 500 opening the substantially cuboid, divided housing assembly 400 adapted to be in an open or close state, the foldable, toy dental chair 450 disposed within the housing assembly 400 when the housing assembly 400 is in a closed state, the toy dental chair 450, when in an unfolded state, configured to seat the doll member 100 when the housing assembly 400 is in an open state. The method further includes step 510 placing the polymer doll member 100, the doll member 100 having at least the body and the head assembly 110, into a substantially inclined position on the toy dental chair member 450, the face portion of the head assembly 110 facing substantially upward. The method further includes step 520 opening the jaw member 120 of the inventive concept which has a distal end of the jaw member 129 and a proximal end of the jaw member 121, the distal end of the jaw member 129 pivotally coupled to the head assembly 110 at a lower front face of the head assembly 130 to create a movable jaw assembly 140.

The method 50 further includes, when the jaw member 120 is in an open state, 530 using at least one or more of the toothbrush 301, tongue scraper 302, and floss tool 303, each with the widened handle 304 adapted to be used by the child to perform cleaning operations to at least simulate cleaning the exposed lower row of protruding teeth members 122 disposed on the inner edge of the proximal edge of the jaw member 124 and the upper row of protruding teeth members 112 disposed on the inner edge of the proximal edge of the lower front face of the head assembly 114 of the jaw member 120, and the tongue member 116 disposed on the upper middle portion of the jaw member 125 and distal portion of the jaw member 129 and interior to the head assembly 110.

FIGS. 14A-14B further illustrate that the method of teaching dental hygiene via the dental hygiene training toy kit 10 further includes step 540 at least one or more of adding and removing the removable hair member 170 couplable to the upper portion of the head assembly 110 via at least one silicone snap member, the snap member 220 having the substantially concave receiving portion 229 disposed on the head assembly 110 or on the hair member 170 and the protruding connector member 221 disposed on the opposing head assembly 110 or hair member 170 adapted to be frictionally coupled within the concave receiving portion 229, the concave receiving portion 229 and the protruding connecting member 221 further being made substantially from silicone.

FIGS. 14A-14B illustrate that the method 60 of teaching dental hygiene via the dental hygiene training toy kit 10 further includes 550 placing the substantially planar mat member 250 to afford the playing surface circumscribed by the raised ridge member 251, the mat member 250 providing the playing surface 255 for the dental hygiene training kit assembly 10, the playing surface 255 further displaying image members 252 directing where to place other members of the kit assembly 10, the playing surface 255 further adapted for the child to play on the mat member with other members of the kit assembly 10.

FIGS. 14A-14B further illustrate that the method 50 of teaching dental hygiene via the dental hygiene training toy kit 10 further includes 560 washing the substantially planar mat member 250 by machine after use. In a related embodiment, toy kit 10 includes a RFID chip for kit location by a parent along with a chip responsive to or in communication with a GPS type applet for locating the user. Kit 10 may also be Bluetooth-enabled or wireless module for communication with a smart device or smartphone applet. The embedded chip may also be responsive to smart speakers to facilitate recognition of child user in the room for more a more enhanced interactive experience. Finally, the training assembly kit and chipset may be paired with a smartphone applet (or a separate remote) to provide wireless prompts to the parent/caregiver and child that simulate the dental office atmosphere with office sounds, an AI receptionist and an AI assistant and other features to assist the child user at play.

The following patents are incorporated by reference in their entireties: U.S. Pat. No. 5,232,370, 20050136384A1, U.S. Pat. No. 4,221,060, and 20080227071A1 along with KR20190003228U and CN212966653U.

While the inventive concept has been described above in terms of specific embodiments, it is to be understood that the inventive concept is not limited to these disclosed embodiments. Upon reading the teachings of this disclosure many modifications and other embodiments of the inventive concept will come to mind of those skilled in the art to which this inventive concept pertains, and which are intended to be and are covered by both this disclosure and the appended claims. It is indeed intended that the scope of the inventive concept should be determined by proper interpretation and construction of the appended claims and their legal equivalents, as understood by those of skill in the art relying upon the disclosure in this specification and the attached drawings.

1. A dental hygiene toy training kit assembly comprising:
a polymer doll member, the doll member having a body, two arms, two legs, and a head assembly;
a jaw member having a distal end and a proximal end, the distal end of the jaw member pivotally coupled to the head assembly at a lower front face of the head assembly to create a movable jaw assembly, the movable jaw assembly adapted to be in an open or closed state, the proximal end of the movable jaw assembly contiguously coupled to a proximal edge of the lower front face of the head assembly, wherein the jaw member when in an open state exposes a lower row of protruding teeth members disposed on an inner edge of the proximal end of the jaw member, and exposes an upper row of protruding teeth members disposed on an inner edge of the proximal edge of the lower front face of the head assembly, and a tongue member disposed on an upper middle and distal portion of the jaw member and interior to the head assembly;
at least one or more of a toothbrush, tongue scraper, and floss tool, each with a widened handle each adapted to be used by the child to perform cleaning operations on the teeth and tongue members; and
a substantially cuboid, divided housing assembly adapted to be in an open or close state, a foldable, toy dental chair disposed within the housing assembly when the housing assembly is in a closed state, the toy dental chair in an unfolded state configured to seat the doll member when the housing assembly is in an open state.

2. The dental hygiene training toy kit assembly of claim 1 wherein a ridged silicone grip member is disposed on an outer surface of the widened handle members of the toothbrush, tongue scraper, and floss tool.

3. The dental hygiene training toy kit assembly of claim 1 wherein a handle portion is pivotally coupled to the housing assembly.

4. The dental hygiene training toy kit assembly of claim 1 wherein a mouthwash bottle is included in the kit assembly.

5. The dental hygiene training toy kit assembly of claim 1 wherein a removable hair member is coupled to an upper portion of the head assembly, the kit adapted to include one or more hair members.

6. The dental hygiene training toy kit assembly of claim 5 wherein the removable hair member is coupled to an upper portion of the head assembly via at least one silicone snap member, the snap member having a substantially concave receiving portion disposed on the head assembly or on the hair member and a protruding connector member disposed on the opposing head assembly or hair member adapted to be frictionally coupled within the concave receiving portion, the concave receiving portion and the protruding connecting member further being made substantially from silicone.

7. The dental hygiene training toy kit assembly of claim 1 wherein a substantially planar mat member affords a playing surface circumscribed by a raised ridge member, the mat member providing a playing surface for the dental hygiene training kit assembly, the playing surface further displaying image members directing where to place other members of the kit assembly, the playing surface further adapted for the child to play on the mat member with other members of the kit assembly.

8. The dental hygiene training toy kit assembly of claim 1 wherein the kit includes a Bluetooth-enabled or wireless module for communication with a smart device or smartphone applet.

9. A dental hygiene toy training kit assembly comprising:
a polymer doll member, the doll member having at least a body member and a head assembly;
a jaw member having a distal end and a proximal end, the distal end of the jaw member pivotally coupled to the head assembly at a lower front face of the head assembly to create a movable jaw assembly, the movable jaw assembly adapted to be in an open or closed state, the proximal end of the movable jaw assembly contiguously coupled to a proximal edge of the lower front face of the head assembly, wherein the jaw member when in an open state exposes a lower row of protruding teeth members disposed on an inner edge of the proximal end of the jaw member, and exposes an upper row of protruding teeth members disposed on an inner edge of the proximal edge of the lower front face of the head assembly, and a tongue member disposed on an upper middle and distal portion of the jaw member and interior to the head assembly;
at least one or more of a toothbrush, tongue scraper, and floss tool, each with a widened handle each adapted to be used by the child to perform cleaning operations on the teeth and tongue members; and
a substantially cuboid, divided housing assembly adapted to be in an open or close state, a foldable, toy dental chair disposed within the housing assembly when the housing assembly is in a closed state, the toy dental chair in an unfolded state configured to seat the doll member when the housing assembly is in an open state.

10. The dental hygiene training toy kit assembly of claim 9 wherein a ridged silicone grip member is disposed on an outer surface of the widened handle members of the toothbrush, tongue scraper, and floss tool.

11. The dental hygiene training toy kit assembly of claim 9 wherein a handle portion is pivotally coupled to the housing assembly.

12. The dental hygiene training toy kit assembly of claim 9 wherein a mouthwash bottle is included in the kit assembly.

13. The dental hygiene training toy kit assembly of claim 9 wherein a removable hair member is coupled to an upper portion of the head assembly, the kit adapted to include one or more hair members.

14. The dental hygiene training toy kit assembly of claim 13 wherein the removable hair member is coupled to an upper portion of the head assembly via at least one silicone snap member, the snap member having a substantially concave receiving portion disposed on the head assembly or on the hair member and a protruding connector member disposed on the opposing head assembly or hair member adapted to be frictionally coupled within the concave receiving portion, the concave receiving portion and the protruding connecting member further being made substantially from silicone.

15. The dental hygiene training toy kit assembly of claim 9 wherein a substantially planar mat member affords a playing surface circumscribed by a raised ridge member, the mat member providing a playing surface for the dental hygiene training kit assembly, the playing surface further displaying image members directing where to place other members of the kit assembly, the playing surface further adapted for the child to play on the mat member with other members of the kit assembly.

16. The dental hygiene training toy kit assembly of claim 9 wherein the kit includes a Bluetooth-enabled or wireless module for communication with a smart device or smartphone applet.

17. A method of teaching dental hygiene via a dental hygiene toy training kit assembly, the method comprising:
opening a substantially cuboid, divided housing assembly adapted to be in an open or close state, a foldable, toy dental chair disposed within the housing assembly when the housing assembly is in a closed state, the toy dental chair in an unfolded state configured to seat the doll member when the housing assembly is in an open state;
placing the polymer doll member, the doll member having at least a body and a head assembly, into a substantially inclined position on the toy dental chair member, a face portion of the head assembly facing substantially upward;
opening a jaw member, the jaw member having a distal end of the jaw member and a proximal end of the jaw member, the distal end of the jaw member pivotally coupled to the head assembly at a lower front face of the head assembly to create a movable jaw assembly; and
when the jaw member is in an open state, using at least one or more of a toothbrush, tongue scraper, and floss tool, each with a widened handle adapted to be used by the child to perform cleaning operations on the teeth and tongue members, to at least simulate cleaning the exposed lower row of protruding teeth members disposed on an inner edge of the proximal edge of the jaw member and an upper row of protruding teeth members disposed on an inner edge of the proximal edge of the lower front face of the head assembly of the jaw member, and a tongue member disposed on an upper middle and distal portion of the jaw member and interior to the head assembly.

18. The method of teaching dental hygiene via a dental hygiene training toy kit of claim 17, the method further including at least one or more of adding and removing a removable hair member couplable to an upper portion of the head assembly via at least one silicone snap member, the snap member having a substantially concave receiving portion disposed on the head assembly or on the hair member and a protruding connector member disposed on the opposing head assembly or hair member adapted to be frictionally coupled within the concave receiving portion, the concave receiving portion and the protruding connecting member further being made substantially from silicone.

19. The method of teaching dental hygiene via a dental hygiene training toy kit of claim 17, the method further including placing a substantially planar mat member to afford a playing surface circumscribed by a raised ridge member, the mat member providing a playing surface for the dental hygiene training kit assembly, the playing surface further displaying image members directing where to place other members of the kit assembly, the playing surface further adapted for the child to play on the mat member with other members of the kit assembly.

20. The method of teaching dental hygiene via a dental hygiene training toy kit of claim 19, the method further including wirelessly communicating with the training kit via a Bluetooth-enabled or wireless module with a smart device or smartphone applet.
